(19) United States
(12) Patent Application Publication
(10) Pub. No.: US 2005/0037382 A1
(43) Pub. Date: Feb. 17, 2005

(54) Methods and compositions for treating and preventing autoimmune disorders

(76) Inventors: Simon C. Robson, Weston, MA (US); Yousif I. A-Rahim, Honolulu, HI (US)

Correspondence Address: CLARK & ELBING LLP, 101 FEDERAL STREET, BOSTON, MA, 02110, US

(21) Appl. No.: 10/820,335

(22) Filed: Apr. 8, 2004

Related U.S. Application Data
(60) Provisional application No. 60/461,160, filed on Apr. 8, 2003.

(51) Int. Cl. C12Q 1/68; G01N 33/53; G01N 33/567

(52) U.S. Cl. 435/6; 435/7.2

(57) ABSTRACT

The invention features methods for reducing, treating, or preventing autoimmune disorders by administering NTDPases, or alternatively, P2 receptor inhibitors to a mammal in need thereof. The invention also features methods for diagnosing a mammal as having or at risk of having an autoimmune disorder by detecting the biological activity of NTPDases or P2 receptors. Also disclosed are screening methods that make use of NTPDases and P2 receptors for the identification of novel therapeutics for autoimmune disorders.

### CROSS REFERENCES TO RELATED APPLICATIONS

This application claims the benefit of the filing date of U.S. Provisional Application, U.S. Ser. No. 60/461,160, filed Apr. 8, 2003.

### FIELD OF THE INVENTION

The field of this invention is the treatment and prevention of autoimmune disorders. In particular, the invention features methods of treating, preventing, or reducing autoimmune disorders by administering NTDPases, or alternatively, P2 receptor inhibitors to a mammal in need thereof. Further, methods are provided for identifying candidate compounds useful for treating or preventing autoimmune disorders.

### BACKGROUND OF THE INVENTION

Autoimmune disorders (AD) typically arise when the immune system (IS) mounts a robust and aberrant response against self-antigens. Due to the wide range of cells that the IS can target, AD are manifested in various ways and can affect, for example, the gastro-intestinal system, the skin, the vascular system, and the nervous system. These disorders are often chronic, requiring lifelong care and monitoring, even when the affected individual appears to be asymptomatic.

Currently, few AD can be treated or “cured” and most affected patients typically receive therapies that can relieve the symptoms associated with autoimmune disorders, at least on a short-term basis. For example, patients suffering from AD are often treated with therapeutic strategies, which strive to minimize the consequences of the inflammatory damage that is caused by AD. In some diseases, such as lupus or rheumatoid arthritis for example, medication can occasionally slow or stop the immune system's destruction of the kidneys or joints. In most cases however, AD are treated using therapies that weaken or suppress the immune response in an attempt to halt the associated inflammation. These drugs are commonly known as immunosuppressive agents and include, for example, corticosteroids (prednisone), methotrexate, cyclophosphamide, azathioprine, and cyclosporin. Unfortunately, these agents also suppress the ability of the immune system to fight infections and have other potentially serious side effects. In some people, a limited number of immunosuppressive agents can result in disease remission although patients are rarely able to stop treatment altogether. The possibility that the disease may resume upon discontinuation must be balanced with the long-term side effects from the immunosuppressive medication.

Thus, better therapeutic modalities are required to reduce or prevent autoimmune disorders.

### SUMMARY OF THE INVENTION

In general, the present invention features methods for treating, reducing, or preventing autoimmune disorders in a mammal in need thereof by administering to the mammal an effective amount of a biologically active NTPDase protein, or alternatively, a P2 receptor inhibitor. Accordingly, the invention is useful to treat, reduce, or prevent autoimmune disorders, such as Addison's disease, alopecia, ankylosing spondylitis, antiphospholipid syndrome, chronic fatigue syndrome, Crohn's disease, ulcerative colitis, diabetes, pemphigus vulgaris, fibromyalgia, Goodpasture syndrome, Graves' disease, idiopathic thrombocytopenic purpura, lupus, Meniere's multiple sclerosis, Behcet's disease, myasthenia gravis, primary biliary cirrhosis, psoriasis, rheumatoid arthritis, rheumatic fever, sarcoidosis, scleroderma, vasculitis, vitiligo, or Wegener's granulomatosis.

In a first aspect, the invention features methods for treating, reducing, or preventing an autoimmune disorder in a mammal in need thereof by administering to the mammal a therapeutically effective amount of an NTPDase protein, a P2 receptor inhibitor, or both.

In another aspect, the invention provides methods for treating, reducing, or preventing an autoimmune disorder in a mammal in need thereof by administering to the mammal a nucleic acid molecule encoding an NTPDase protein, a P2 receptor inhibitor, or both in a therapeutically effective amount.

In yet another aspect, the present invention provides methods for treating, reducing, or preventing an autoimmune disorder in a mammal in need thereof by administering to the mammal a therapeutically effective amount of a compound that increases the biological activity of an NTPDase. Alternatively, the mammal may be administered with a compound that reduces the biological activity of a P2 receptor. In either case, the compound may be a nucleotide analog, a peptide, an antibody, an oligonucleotide or analog thereof, a natural compound, or a synthetic compound.

In another aspect, the invention also features a method of diagnosing a mammal having or at risk of having an autoimmune condition. Mammals at risk of having or having an autoimmune disorder are characterized by a reduction in the biological activity of an NTPDase, by an increase in the biological activity of a P2 receptor, or both. Such biological activity may be assessed by measuring the biological activity of the NTPDase or P2 receptor, or alternatively by measuring mRNA or protein levels of the NTPDase or the P2 receptor.

In all foregoing aspects of this invention, the mammal being treated is preferably a human patient and autoimmune disorders treated according to the present invention include any condition that is characterized by an immune response mounted to a self-antigen. In some cases, the autoimmune disorder may also have an inflammatory component. Exemplary autoimmune diseases include, for example, Addison's disease, alopecia, ankylosing spondylitis, antiphospholipid syndrome, Behcet's disease, chronic fatigue syndrome, Crohn's disease, ulcerative colitis, diabetes, fibromyalgia, Goodpasture syndrome, Graves' disease, idiopathic thrombocytopenic purpura, lupus, Meniere's multiple sclerosis, myasthenia gravis, pemphigus vulgaris, primary biliary cirrhosis, psoriasis, rheumatoid arthritis, rheumatic fever, sarcoidosis, scleroderma, vasculitis, vitiligo, or Wegener's granulomatosis.

According to this invention, the NTPDase may be, for example, CD39, CD39L1, CD39L2, CD39L3, CD39L4, potato apyrase, hepatic canlicular ecto-apyrase, Golgi-associated ecto-ATPase, ecto-uridine diphosphatase (UDPase), lysosomal ecto-apryase LAP70, or α-sarcoglycan. Preferably, the NTPDase is CD39. The P2 receptor inhibitor is any agent that reduces the biological activity of a P2 receptor (e.g., P2Y6, P2Y4, P2Y12, P2Y5, P2Y10, P2Y11, P2Y1, P2Y2, P2X1, P2X4, or P2X7) and includes, for example, suramin, TNP-ATP, KN-62, MRS2179, TNP-GTP, PPADS, oxidized ATP, or Reactive Blue2. If desired, more than one NDTPase, or more than one P2 receptor inhibitor may be administered to the mammal being treated.

If desired, the mammal administered with the NDTPase or the P2 receptor inhibitor may also receive a second therapeutic regimen, which may include a therapeutic agent. Examples of second therapeutic agents include immunosuppressors (e.g., azathioprine, 6-mercaptopurine, cyclosporine A, tacrolimus, cyclophosphamide, or methotrexate), anti-inflammatory agents (e.g., sulfasalazine, olsalazine, mesalmine, or non-steroidal anti-inflammatory agents such as ibuprofen, ketoprofen, piroxicam, naproxen sodium, sulindac, aspirin, choline subsalicylate, diflunisal, oxaproxin, etodolac, ketorolac, fenoprofen, flurbiprofen, indomethacin, fenamates, meclofenamate, mefenamic acid, nabumetone, oxicam, piroxicam, salsalate, tolmetin, and magnesium salicylate), or steroids (e.g., cortisone, budesonide, or prednisone).

In another aspect, the invention provides a method for identifying a candidate compound for treating, reducing, or preventing an autoimmune disorder in a mammal. The method involves the steps of: (a) contacting a cell expressing an NTPDase gene with a candidate compound; and (b) measuring NTPDase gene expression or NTPDase protein activity in the cell. A candidate compound that increases the expression or the activity of NTPDase, relative to NTPDase expression or activity in a cell not contacted with the candidate compound, is identified as useful for treating, reducing, or preventing an autoimmune disorder in a mammal.

In preferred embodiments, the NTPDase gene is an NTPDase fusion gene. In other embodiments, step (b) involves the measurement of NTPDase MRNA or protein. Desirably, the NTPDase is CD39.

In yet another aspect, the invention provides a method for identifying a candidate compound for treating, reducing, or preventing an autoimmune disorder in a mammal. The method involves the steps of: (a) contacting a cell expressing a P2 receptor gene with a candidate compound; and (b) measuring P2 receptor gene expression or P2 receptor protein activity in the cell. A candidate compound that reduces the expression or the activity of P2 receptor, relative to P2 receptor expression or activity in a cell not contacted with the candidate compound, is identified as useful for treating, reducing, or preventing an autoimmune disorder in a mammal.

In preferred embodiments, the P2 receptor gene is a P2 receptor fusion gene. In other embodiments, step (b) involves the measurement of P2 receptor MRNA or protein.

In a related aspect, the invention provides another method for identifying a candidate compound for treating, reducing, or preventing an autoimmune disorder in a mammal. This method involves the steps of: (a) contacting NTPDase protein with a candidate compound; and (b) determining whether the candidate compound binds the NTPDase protein and increases NTPDase activity. Candidate compounds that bind and increase NTPDase activity are identified as useful for treating, reducing, or preventing an autoimmune disorder in a mammal.

In preferred embodiments, the method also tests the ability of the candidate compound to increase the expression of the NTPDase gene in a cell, for example a mammalian cell, such as a rodent or human cell. Most preferably, the NTPDase is human NTPDase. Desirably, the NTPDase is CD39.

In another related aspect, the invention provides another method for identifying a candidate compound for treating, reducing, or preventing an autoimmune disorder in a mammal. This method involves the steps of: (a) contacting a P2 receptor with a candidate compound; and (b) determining whether the candidate compound binds the P2 receptor and reduces P2 receptor activity. Candidate compounds that bind and reduce P2 receptor activity are identified as useful for treating, reducing, or preventing an autoimmune disorder in a mammal.

In preferred embodiments, the method also tests the ability of the candidate compound to reduce the expression of the P2 receptor gene in a cell, for example a mammalian cell such as a rodent or human cell. Most preferably, the P2 receptor is human P2 receptor.

In yet another aspect, the invention provides a kit containing (a) an NTPDase protein; and (b) instructions for delivery of the protein to a mammal under conditions suitable for treating, reducing, or preventing an autoimmune disorder.

The invention also provides a kit containing (a) a vector expressing a nucleic acid encoding an NTPDase protein; and (b) instructions for delivery of the vector to a mammal under conditions suitable for treating, reducing, or preventing an autoimmune disorder.

The invention also provides a kit containing (a) a CD39 protein; and (b) instructions for delivery of the CD39 protein to a mammal under conditions suitable for treating, reducing, or preventing an autoimmune disorder.

The invention also provides a kit containing (a) a vector expressing a nucleic acid encoding a CD39 protein; and (b) instructions for delivery of the vector to a mammal under conditions suitable for treating, reducing, or preventing an autoimmune disorder.

In yet another aspect, the invention provides a kit containing (a) a P2 receptor inhibitor; and (b) instructions for delivery of the inhibitor to a mammal under conditions suitable for treating, reducing, or preventing an autoimmune disorder.

The invention also provides a kit containing (a) a vector expressing a nucleic acid encoding a P2 receptor inhibitor; and (b) instructions for delivery of the vector to a mammal under conditions suitable for treating, reducing, or preventing an autoimmune disorder.

As used herein, by “NTPDase” is meant any polypeptide that exhibits an activity common to its related, naturally occurring NTPDase. Accordingly, the NTPDase of the invention is substantially identical to the naturally occurring NTPDase, and desirably, the NTPDase has an increase of biological activity of at least 1-fold, 2-fold, 3-fold, 4-fold, 5-fold, 10-fold, 20-fold, 50-fold, 100-fold, or more than 100-fold over the activity of the naturally-occurring NTPDase. Preferably, the NTPDase can hydrolyse a nucleotide triphosphate (NTP) into a nucleotide diphosphate (NDP) and inorganic phosphate or into nucleotide monophosphate (NMP) and pyrophosphate. Alternatively, the NTPDase can also hydrolyse NDP into NMP and inorganic phosphate. Preferably, such phosphohydrolysis is at least 5%, 10%, 20%, 30%, 40%, 50%, 60%, 70%, 80%, 90%, or more than 100% above control levels as measured any standard technique in the art used to measure such hydrolysis, including for example assays detecting, measuring, or quantifying the release of one or more reaction products (e.g., NTP, NDP, NMP, pyrophosphate, or inorganic phosphate). Alternatively, the NTPDase of the invention may also decrease the autoimmune responses in the autoimmune disorder by at least 5%, 10%, 20%, 30%, 40%, 50%, 60%, 70%, 80%, 90%, or more than 100% below untreated control levels as measured by any standard technique in the art. Thus, by “NTPDase biological activity” is meant any of the above activities. The NTPDases of the invention include, for example, CD39, CD39L1, CD39L2, CD39L3, CD39L4, potato apyrase, hepatic canlicular ecto-apyrase, Golgi-associated ecto-ATPase, ecto-uridine diphosphatase (UDPase), lysosomal ecto-apryase LAP70, or α-sarcoglycan. Preferably, the NTPDase is CD39.

By “P2 receptor inhibitor” is meant any compound that reduces the biological activity of a P2 receptor (e.g., P2Y6, P2Y12, P2Y1, P2Y2, P2X1, P2X4, or P2X7) by at least 5%, 10%, 20%, 30%, 40%, 50%, 60%, 70%, 80%, 90%, or more than 100% below control levels as measured any standard technique in the art. Alternatively, the activity of the P2 receptor inhibitor of the invention may also decrease the autoimmune response of an autoimmune disorder by at least 5%, 10%, 20%, 30%, 40%, 50%, 60%, 70%, 80%, 90%, or more than 100% below untreated control levels as measured by any standard technique in the art. Exemplary P2 receptor inhibitors include suramin, KN-62, MRS2179, TNP-ATP, TNP-GTP, oxidized ATP, PPADS, or Reactive Blue2.

By “an effective amount” is meant an amount of a compound, alone or in a combination, required to treat, reduce, or prevent an autoimmune disorder in a mammal. Desirably, an effective amount of a compound is any amount of the compound that can increase the biological activity of the NTPDase by at least 5%, 10%, 20%, 30%, 40%, 50%, 60%, 70%, 80%, 90%, or more than 100% above control levels. Alternatively, an effective amount of a compound is any amount of the compound that can reduce the biological activity of the P2 receptor by at least 5%, 10%, 20%, 30%, 40%, 50%, 60%, 70%, 80%, 90%, or more than 100% below control levels. The effective amount of active compound(s) varies depending upon the route of administration, age, body weight, and general health of the subject. Ultimately, the attending physician or veterinarian will decide the appropriate amount and dosage regimen.

By a “candidate compound” is meant a chemical, be it naturally-occurring or artificially-derived, that is tested for its ability to increase NTPDase expression or activity, or alternatively, to decrease P2 receptor expression or activity. Candidate compounds may include, for example, peptides, polypeptides, synthetic organic molecules, naturally occurring organic molecules, nucleic acid molecules, and components thereof.

By “autoimmune disorder” is meant any pathological condition characterized by an immune response mounted against at least one self-antigen. Such conditions include, for example, Addison's disease, alopecia, ankylosing spondylitis, antiphospholipid syndrome, Behcet's disease, chronic fatigue syndrome, Crohn's disease, ulcerative colitis, diabetes, fibromyalgia, Goodpasture syndrome, Graves' disease, idiopathic thrombocytopenic purpura, lupus, Meniere's multiple sclerosis, myasthenia gravis, pemphigus vulgaris, primary biliary cirrhosis, psoriasis, rheumatoid arthritis, rheumatic fever, sarcoidosis, scleroderma, vasculitis, vitiligo, or Wegener's granulomatosis. An autoimmune disorder can be diagnosed by any method known in the art, including for example, examination of blood samples to detect the presence of autoantibodies or autoimmune cells.

By “increases expression of an NTPDase gene or activity of a NTPDase protein” is meant to increase expression or activity of NTPDase relative to control conditions. This increase may be, for example, an increase of least 2-fold, 3-fold, 5-fold, 10-fold, 100-fold, or even 1000-fold or greater, relative to control conditions.

By “decreases expression of a P2 receptor gene or activity of a P2 receptor” is meant to decrease expression or activity of P2 receptor relative to control conditions. This decrease may be, for example, a decrease of least 2-fold, 3-fold, 5-fold, 10-fold, 100-fold, or even 1000-fold or greater, relative to control conditions.

The term “isolated DNA” is meant DNA that is free of the genes which, in the naturally-occurring genome of the organism from which the given DNA is derived, flank the DNA. Thus, the term “isolated DNA” encompasses, for example, cDNA, cloned genomic DNA, and synthetic DNA.

By “NTDPase fusion gene” is meant an NTPDase promoter and/or all or part of an NTPDase coding region operably linked to a second, heterologous nucleic acid sequence. Similarly, by “P2 receptor inhibitor fusion gene” is meant a P2 receptor inhibitor promoter and/or all or part of a P2 receptor inhibitor coding region operably linked to a second, heterologous nucleic acid sequence. In preferred embodiments, the second, heterologous nucleic acid sequence is a reporter gene, that is, a gene whose expression may be assayed; reporter genes include, without limitation, those encoding glucuronidase (GUS), luciferase, chloramphenicol transacetylase (CAT), green fluorescent protein (GFP), alkaline phosphatase, and β-galactosidase.

By “treating, reducing, or preventing an autoimmune disorder” is meant ameliorating such a disorder before or after it has occurred. As compared with an equivalent untreated control, such reduction or degree of prevention is at least 5%, 10%, 20%, 40%, 50%, 60%, 80%, 90%, 95%, or 100% as measured by any standard technique. A patient who is being treated for an autoimmune disorder is one who a medical practitioner has diagnosed as having such a condition. Diagnosis may be by any suitable means. Diagnosis and monitoring may employ, for example, an antinuclear antibody Test, blood test including those that detect self-antibodies, autoimmune cells, or both; family history; biopsy; or X-rays, for example. A patient in whom the development of an autoimmune disorder is being prevented is one who has not received such a diagnosis. One in the art will understand that these patients may have been subjected to the same standard tests as described above or may have been identified, without examination, as one at high risk due to the presence of one or more risk factors (e.g., family history). Thus, prophylactic administration of NTPDase or a P2 receptor inhibitor is considered to be preventing the development of an autoimmune disorder.

By “substantially identical” is meant a polypeptide or nucleic acid exhibiting at least 75%, but preferably 85%, more preferably 90%, most preferably 95%, or even 99% identity to a reference amino acid or nucleic acid sequence. For polypeptides, the length of comparison sequences will generally be at least 20 amino acids, preferably at least 30 amino acids, more preferably at least 40 amino acids, and most preferably 50 amino acids. For nucleic acids, the length of comparison sequences will generally be at least 60 nucleotides, preferably at least 90 nucleotides, and more preferably at least 120nucleotides.

The present invention provides significant advantages over standard therapies for the treatment and prevention of autoimmune disorders. Administration of an NTPDase or a P2 receptor inhibitor according to the present invention reduces the immune responses against self-antigens, thus treating and preventing autoimmune disorders. In addition, the candidate compound screening methods provided by this invention allow for the identification of novel therapeutics that modify the injury process, rather than merely mitigating the symptoms.

### BRIEF DESCRIPTION OF DRAWINGS

FIG. 1A shows a series of pictures representing tissues stained with Haematoxylin and Eosin that were derived from uninvolved colonic tissues (serving as control normal tissues), or patients having ulcerative colitis (UC) or Crohn's disease (CD).

FIG. 1B is an immunoblot detecting CD39 levels in normal patients (NL), or patients having UC or CD.

FIG. 1C is a graph showing the mRNA levels of CD39 from normal colonic tissues, and tissues obtained from patients having Crohn's disease or Ulcerative colitis.

FIG. 1D is a series of immunostains of tissues obtained from uninvolved colonic tissues (serving as control normal tissues) or from patients having UC or CD to detect CD39 expression.

FIG. 2A shows a series of photographs of CD39−/− mice.

FIG. 2B shows a series of gel pictures to validate the CD39 null phenotype of CD39−/− mice as determined by PCR, Southern, and Western analyses.

FIG. 3A shows a graph representing the percentage of weight loss of wild-type CD39+/+, hemizygous CD39+/−, and CD39−/− null mice following acute treatment with dextran sulphate sodium (DSS).

FIG. 3B shows a graph representing the percentage of weight loss of wild-type CD39+/+ mice following chronic treatment with DSS over a period of five weeks.

FIG. 4 shows a graph representing the percentage of hematocrit in CD39+/+ mice treated and not treated with DSS, as well as CD39−/+ and CD39−/− mice treated with DSS.

FIG. 5A is a series of Haematoxylin and Eosin stains representing various stages of mucosal injury which have been graded using a scale of 0 to 4.

FIG. 5B is a series of Haematoxylin and Eosin stains of tissues derived from CD39+/+, CD39+/− mice and CD39−/− mice following acute treatment with DSS.

FIG. 6A is an immunostain of colonic tissue from CD39+/+ mice to detect CD39 expression.

FIG. 6B is an immunostain of colonic tissue from CD39+/+ mice to detect CD31 expression.

FIG. 6C is an immunostain of colonic tissue from CD39+/+ mice to detect fibrin.

FIG. 6D is an immunostain of colonic tissue from CD39+/+ mice to detect macrophages.

FIG. 6E is an immunostain of colonic tissue from CD39+/+ mice to detect P-selectin expression.

FIG. 7A is an immunostain of colonic tissue from CD39+/− mice to detect CD39 expression.

FIG. 7B is an immunostain of colonic tissue from CD39+/− mice to detect CD31 expression.

FIG. 7C is an immunostain of colonic tissue from CD39+/− mice to detect fibrin.

FIG. 7D is an immunostain of colonic tissue from CD39+/− mice to detect macrophages.

FIG. 7E is an immunostain of colonic tissue from CD39+/− mice to detect P-selectin expression.

FIG. 8A is an immunostain of colonic tissue from CD39−/− mice to detect CD31 expression.

FIG. 8B is an immunostain of colonic tissue from CD39−/− mice to detect macrophages.

FIG. 9A shows a graph of the weight (g) of CD39+/+ mice treated chronically with DSS and administered at week 5 with 0.4 units/g of apyrase.

FIG. 9B shows a graph of the percentage of weight change in CD39+/− mice treated chronically with DSS and administered at week 5 with 0.4 units/g of apyrase.

### DETAILED DESCRIPTION

In general, the present invention features methods for treating, reducing, or preventing an autoimmune condition in a mammal in need thereof by providing the mammal an effective amount of a biologically active NTPDase protein, or alternatively, a P2 receptor inhibitor at the same levels as obtained when increasing the biological activity of NTPDases. Accordingly, the invention is particularly useful to treat, reduce, or prevent autoimmune conditions, such as Addison's disease, alopecia, ankylosing spondylitis, antiphospholipid syndrome, Behcet's disease, chronic fatigue syndrome, Crohn's disease, ulcerative colitis, diabetes, fibromyalgia, Goodpasture syndrome, Graves' disease, idiopathic thrombocytopenic purpura, lupus, Meniere's multiple sclerosis, myasthenia gravis, pemphigus vulgaris, primary biliary cirrhosis, psoriasis, rheumatoid arthritis, rheumatic fever, sarcoidosis, scleroderma, vasculitis, vitiligo, or Wegener's granulomatosis. In some cases, such autoimmune disorders also have an inflammatory component.

The Role of CD39 and P2 receptors in Immune Responses

Extracellular nucleotides released in the blood following arterial vascular injury for example, are known to influence the activity of purinergic/pyrimidinergic type-2 (P2) receptors, namely the ligand gated ion channel P2X receptors and the G-protein coupled P2Y receptors. This interaction results in the activation of platelets, endothelial cells, monocytes/macrophages, and leukocytes in turn modulating cardiac function, vasomotor responses, platelet activation, and thrombosis, as well as inflammatory and immune processes. The ectonucleotidases of the nucleoside triphosphate diphosphohydrolase (NTPDase)/CD39 family, are Ca²⁺/Mg²⁺-dependent ectoenzymes that also participate in this process by hydrolyzing nucleoside 5′-triphosphates and nucleoside 5′-diphosphates. Since these ectoenzymes bind and hydrolyze extracellular ATP (and ADP) to produce AMP, for example, an important function of these enzymes is the modulation of P2-receptor-mediated signaling by the removal of extracellular ATP and ADP, as well as related nucleotides. The ultimate generation of extracellular adenosine not only abrogates or terminates nucleotide-mediated effects, but also activates adenosine receptors, with often opposing (patho)physiological effects. The regulated dephosphorylation of extracellular nucleotides by ectonucleotidases is therefore critical for appropriate purinergic/pyrimidinergic signaling and metabolic homeostasis.

The present invention is based on the discovery that an increase in the biological activity of NTPDases such as CD39, or conversely, a reduction in the biological activity of P2 receptors reduces the immune response in autoimmune conditions (e.g., ulcerative colitis and Crohn's disease) in human and mice models. Thus, according to this invention, the administration of an NTPDase, such as CD39, or a P2 receptor inhibitor can modulate the immune response in a mammal such that the autoimmune disorder is treated, reduced or prevented.

Pharmaceutical Compositions

NTPDase

According to the present invention, the administration of a biologically active NTPDase to a mammal leads to the phosphohydrolysis of nucleotides. NTPDases include, for example, CD39, CD39L1, CD39L2, CD39L3, CD39L4, potato apyrase, hepatic canlicular ecto-apyrase, Golgi-associated ecto-ATPase, ecto-uridine diphosphatase (UDPase), lysosomal ecto-apryase LAP70, or α-sarcoglycan. Desirably, the NTPDase is CD39. The preferred biologically active dose of NTPDase within the practice of the present invention is a dosing that will induce the maximum hydrolysis of nucleotides and reduction in autoimmune responses. Thus, a biologically active NTPDase according to this invention is substantially identical to the naturally occurring NTPDase, and has the ability to increase hydrolysis of nucleotides (NTP or NDP) by at least 5%, 10%, 20%, 30%, 40%, 50%, 60%, 70%, 80%, 90%, or more than 100% above untreated control levels, as measured any standard technique in the art used to measure such hydrolysis. Desirably, the NTPDase of the invention, has an increase of biological activity of at least 1-fold, 2-fold, 3-fold, 4-fold, 5-fold, 10-fold, 20-fold, 50-fold, 100-fold, or more than 100-fold over the activity of the naturally-occurring NTPDase. NTPDase activity may be measured by any standard technique in the art, such as assays detecting, measuring, or quantifying the release of one or more reaction products (e.g., NTP, NDP, NMP, pyrophosphate, or inorganic phosphate) and described, for example, by Lust et al. (Anal. Biochem. (1981) 110(2): 258-266), hereby incorporated by reference. Alternatively, the biological activity of NTPDase can be measured by assessing its ability to reduce autoimmune responses by at least by at least 5%, 10%, 20%, 30%, 40%, 50%, 60%, 70%, 80%, 90%, or more than 100% below untreated control levels using standard techniques in the art. According to this invention, autoimmune responses may be measured, for example, by the detection of autoimmune antibodies and autoimmune cells. Since autoimmune responses are also characterized by inflammation, autoimmune responses may also detect the presence of inflammatory cells, such as infiltrating leucocytes (e.g., by immunohistochemistry), the release of pro-inflammatory molecules (e.g., by immunohistochemistry, Northern analysis, Western analysis, or RT-PCR), or the activation of inflammatory signaling pathway (e.g., activation of the NF-κB pathway). The NTDPase of the present invention is therefore any agent having any one or more of these activities.

P2 Receptor Inhibitors

Two main families of P2 receptors are currently known, namely the P2X receptor and the P2Y receptor. According to this invention, a P2 receptor inhibitor reduces the activity of at least one P2X receptor, P2Y receptor, or both. Such P2 receptors include for example, P2Y6, P2Y4, P2Y12, P2Y5, P2Y10, P2Y11, P2Y1, P2X1, P2X4, or P2X7. Preferably, the P2 receptor inhibitor reduces the biological activity of the P2Y6, P2Y12, P2Y1, P2Y2, P2X1, P2X4, or P2X7. Desirably, the activity of the P2 receptor following treatment with the P2 receptor inhibitor is reduced by at least 5%, 10%, 20%, 30%, 40%, 50%, 60%, 70%, 80%, 90%, or more than 100% below untreated control levels using standard techniques in the art. For example, the reduction of the biological activity of P2 receptor can be measured by measuring the reduction in the autoimmune response using any of the techniques described above or alternatively, the ability of P2 receptor inhibitor to treat, reduce, or prevent autoimmune conditions may be assessed by measuring P2 receptor mRNA or protein levels using any standard technique in the art.

Second Therapeutic Agents

According to the present invention, the NTPDase or the P2 receptor inhibitor is delivered in the mammal in a pharmaceutically acceptable carrier, alone or in combination with one or more therapeutic agents. An NTPDase may also be administered in combination with a P2 receptor inhibitor. When the second therapeutic agent is present in a different pharmaceutical composition, different routes of administration may be used. For example, the second therapeutic agent may be administered orally, or by intravenous, intramuscular, or subcutaneous injection. If desired, more than one therapeutic agent may be administered with the NTPDase or the P2 receptor inhibitor and concentrations known to be effective for such therapeutic agents can be used. Desirably, the NTPDase or the P2 receptor inhibitor in combination with the secondary therapeutic agent is administered in a single pharmaceutical composition consisting of an effective amount in a pharmaceutically acceptable carrier. Alternatively, the NTPDase (or the P2 receptor inhibitor) of the invention and the second therapeutic agent are administered in separate formulations within at least 1, 2, 4, 6, 10, 12, 18, 24 hours or more than 24 hours apart. These reagents can be combined and used with additional active or inert ingredients, e.g., in conventional pharmaceutically acceptable carriers. A pharmaceutical carrier can be any compatible, non-toxic substance suitable for the administration of the compositions of the present invention to a mammal. Pharmaceutically acceptable carriers include for example water, saline, buffers and other compounds described for example in the Merck index Merck & co. Rahway, N.J. Slow release formulation or a slow release apparatus may be also be used for continuous administration.

Second therapeutic agents may include for example steroids (e.g., cortisone, budesonide, or prednisone), immunosuppressors (e.g., azathioprine, 6-mercaptopurine, cyclosporine A, tacrolimus, cyclophosphamide, or methotrexate), or anti-inflammatory agents (e.g., sulfasalazine, olsalazine, mesalmine, or a non-steroidal anti-inflammatory agent such as ibuprofen, ketoprofen, piroxicam, naproxen sodium, sulindac, aspirin, choline subsalicylate, diflunisal, oxaproxin, etodolac, ketorolac, fenoprofen, flurbiprofen, indomethacin, fenamates, meclofenamate, mefenamic acid, nabumetone, oxicam, piroxicam, salsalate, tolmetin, and magnesium salicylate). Concentrations of NTPDase (or the P2 receptor inhibitor) and the second therapeutic agent necessary to reduce an autoimmune response will depend upon different factors, including means of administration, target site, physiological state of the mammal, and other medication administered. Thus treatment dosages may be titrated to optimize safety and efficacy and is within the skill of an artisan of the art. Determination of the proper dosage and administration regime for a particular situation is within the skill of the art.

Formulation and Routes of Administration

According to the invention, the NTPDase (or P2 receptor) may be administered to the mammal by means of expression vectors containing a nucleic acid sequence encoding for a biologically active NTPDase (or P2 receptor), respectively, substantially identical to the naturally occurring NTPDase (or P2 receptor). The NTPDase (or P2 receptor inhibitor) is therefore a polypeptide or nucleic acid exhibiting at least 75%, but preferably 85%, more preferably 90%, most preferably 95%, or even 99% identity to a reference amino acid or nucleic acid sequence of the naturally occurring NTPDase (or P2 receptor inhibitor). For polypeptides, the length of comparison sequences will generally be at least 20 amino acids, preferably at least 30 amino acids, more preferably at least 40 amino acids, and most preferably 50 amino acids. For nucleic acids, the length of comparison sequences will generally be at least 60 nucleotides, preferably at least 90 nucleotides, and more preferably at least 120 nucleotides. An expression vector of this invention may be in any of several forms, including, but not limited to, RNA, DNA, DNA encapsulated in an adenovirus coat, DNA packaged in another viral or viral-like form (such as herpes simplex, and AAV), DNA encapsulated in liposomes, DNA complexed with polylysine, complexed with synthetic polycationic molecules, conjugated with transferrin, complexed with compounds such as PEG to immunologically “mask” the molecule and/or increase half-life, or conjugated to a non-viral protein. Preferably, the polynucleotide is DNA and includes not only bases A, T, C, and G, but also includes any of their analogs or modified forms of these bases, such as methylated nucleotides, intemucleotide modifications such as uncharged linkages and thioates, use of sugar analogs, and modified and/or alternative backbone structures, such as polyamides. Alternatively, the NTPDase protein (or P2 receptor inhibitor) may be directly administered to cells of a mammal using for example microinjection techniques.

Typically, administration of plasmids encoding the NTPDase (or P2 receptor inhibitor) into a mammal comprise about 1 nanogram to about 5000 micrograms of DNA. Desirably, compositions comprise about 5 nanograms to 1000 micrograms of DNA, 10 nanograms to 800 micrograms of DNA, 0.1 micrograms to 500 micrograms of DNA, 1 microgram to 350 micrograms of DNA, 25 micrograms to 250 micrograms of DNA, or 100 micrograms to 200 micrograms of DNA. Alternatively, administration of recombinant adenoviral vectors encoding the NTPDase (or P2 receptor inhibitor) into a mammal may be administered at a concentration of at least 10⁵, 10⁶, 10⁷, 10⁸, 10⁹, 10¹⁰, or 10¹¹ plaque forming unit (pfu). The pharmaceutical compositions according to the present inventions are formulated according to the mode of administration to be used. In cases where pharmaceutical compositions are injectable pharmaceutical compositions, they are sterile, pyrogen-free and particulate free. An isotonic formulation is preferably used. Generally, additives for isotonicity can include for example sodium chloride, dextrose, mannitol, sorbitol and lactose. Stabilizers may also be used and include, for example, gelatin and albumin.

Overall, the pharmaceutical composition including the NTPDase (or P2 receptor inhibitor) of the invention can be provided by injection (e.g., intrasmuscular, intranasal, intraperitoneal, intradermal, subcutaneous, intravenous, intraarterial, or intraoccular), as well as by oral, topical (e.g., ointment, foam, or patch), or transdermal administration. Typically, these compositions may be provided by means of suppositories or enemas. Compositions according to the invention may also be provided to mucosal tissue, by lavage to the rectal or dermal tissue, for example.

Patients Amenable to Treatment

According to this invention, the administration of the NTPDase (or P2 receptor inhibitor) of the invention is useful to treat, reduce, or prevent an autoimmune disorder (e.g., intestinal bowel disease such as Crohn's disease or ulcerative colitis) in a mammal, preferably a human patient. Such conditions are diagnosed by any standard technique in the art, including for example, the examination of blood tests for the presence of autoantibodies or autoimmune cells, X-rays, family history, or any method that has been developed to diagnose a particular autoimmune disorder. In the case of Crohn's disease and ulcerative colitis, for example, diagnosis is typically based on sigmoidoscopy, by barium enema X-ray, by colonoscopy, or upper gastro-intestinal endoscopy; alternatively, diagnosis may also be performed based on examination of stools or blood samples.

Typically, an autoimmune disorder has been treated or prevented if symptoms or damage inflicted by autoimmune responses are reduced by reduced by at least 5%, 10%, 20%, 30%, 40%, 50%, 60%, 70%, 80%, 90%, more than 90%, or even 100% as measured by any standard technique. A patient in whom the development of an autoimmune disorder is being prevented is one who has not received such a diagnosis according to such techniques. One in the art will understand that these patients may have been subjected to the same tests or may have been identified, without examination, as one at high risk due to the presence of one or more risk factors (e.g., family history, quality of nutrition, presence of molecular markers of autoimmune disorders, presence of autoantibodies or autoimmune cells, age, race, or sex). Reduction of autoimmune symptoms or damage may also include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Treatment may occur at home with close supervision by the health care provider, or may occur in a health care facility.

Determination of Inflammation

Because inflammation is often concomitant with autoimmune responses, autoimmune responses may also be assessed by detecting inflammation using any method known in the art, by detecting for example, pro-inflammatory markers, the release of pro-inflammatory molecules (e.g., TNF-alpha, IL-1 beta, IL-6, IL-10, GRO-CINC-1, IL-5, IL-18 or MCP-1), or the activation of pro-inflammatory signaling. The detection of such molecules may be determined both in vitro (by Western or Northern analysis, for example) or in vivo (as measured by immunohistochemical methods). Furthermore, inflammation may also be determined using assays, which measure myeloperoxidase activity, which is an indication of acute inflammation for example. Alternatively, overall morphology of tissues and the detection of infiltration of pro-inflammatory cells such as leukocytes, monocytes, macrophages (F4/80, or ER-MP20 for example), lymphocytes (IgA, IgG, IgM, CD4 and CD8 staining), neutrophils, and eosinophils (by immunohistochemical methods) also demonstrate the presence of inflammation.

Identification of a Candidate Compound that Modulates the Biological Activity of NTPDases or P2 Receptors

Candidate compounds that can specifically modulate the biological activity of NTPDases or P2 receptors and can thereby treat, reduce, or prevent autoimmune disorder, can be identified by the methods of the invention. Such methods have previously been described by U.S. Patent Application and PCT US02/40471, both hereby incorporated by reference. The candidate compound is identified for its usefulness in the treatment, reduction, or prevention of autoimmune disorders and is identified by its ability to increase the biological activity of a NTPDase, such as CD39, or the expression level of an CD39 gene as determined by any standard method in the art. Alternatively, a candidate compound is identified using the same techniques by its ability to decrease the biological activity or expression levels of a P2 receptor. According to the invention, the biological activity of an NTPDase is increased, for example, if it decreases the phosphohydrolysis of nucleoside di- and tripohosphates. A candidate compound of the invention can also, for example, increase the expression of an NTPDase gene by increasing transcription of the NTPDase gene, or translation or stability of the NTPDase mRNA.

Desirable candidate compounds include, for example, nucleotide analogs, peptides (for example a NTPDase or fragment thereof, see Gangadharan et al., Surgery 130:296-303 (2001), hereby incorporated by reference), antibodies, antisense or oligonucleotide analogs (see, Imai et al., (1999) Biochemistry 38:13473-13479, hereby incorporated by reference), natural compounds, and synthetic compounds. Other molecules that modulate NTPDase biological activity, such as molecules related to the suramin-Evans blue families, can also be identified by the methods of the invention. Methods for screening such compounds are provided in detail in U.S. Patent Application Ser. No. 60/441,905 and PCT US02/40471.

Diagnosis of an Increased Risk of an Autoimmune Disorder

Based on our discovery that the expression of NTPDases, such as CD39, can suppress the autoimmune and inflammatory responses in autoimmune disorders by its modulatory function on nucleotide-sensitive P2 receptors on inflammatory cells, the present invention also provides diagnostic assays to predict or diagnose a mammal, such as a patient, as having or at risk of having an autoimmune disorder. Thus, a reduction in CD39 expression or biological activity levels in a mammal (e.g., a reduction in CD39 mRNA or polypeptide levels, or the phosphohydrolysis of nucleoside di- or triphosphates mediated by NTPDases) relative to a control individual diagnosed as not having such a condition, would indicate that a subject has an increased risk of developing, or has such a condition. One of skill in the art will appreciate that many different types of diagnostic assays can be used to detect such a reduction in NTPDase activity or expression. Similarly, diagnostic assays may also be designed which detect an increase in the biological activity of a P2 receptor to diagnose a mammal as having or at increased risk of having an autoimmune disorder. Such diagnostic assays are described, for example, in U.S. patent application Ser. No. 60/441,905 and PCT US02/40471, both hereby incorporated by reference.

NTPDase Activity Measurement

Enzyme activity in protein fractions was determined as follows (Sevigny J et al. (1997) Biochim Biophys Acta 1334(1): 73-88). Following the addition of protein samples to a buffer containing 1 mL of 8 mM CaCl2, 50 mM Tris, and 50 mM imidazole, pH 7.4, the mixture was preincubated at 37° C. for 3 minutes. The enzyme reaction was initiated by the addition of 0.3 mM substrate (ATP or ADP) and terminated at 5 to 15 minutes with 0.25 mL of the malachite green reagent, after which, the resulting inorganic phosphate released from exogenous nucleotides was measured. To determine specific activities, the protein content of the enzyme preparations was measured using the Bradford technique.

Antibodies

Anti-murine NTPDase 1 and NTPDase 2 polyclonal antibodies were raised in rabbits by direct intramuscular and subcutaneous injection of cDNA encoding the whole gene ligated into pcDNA3. Plasmids expressing mouse NTPDase 1 and the open reading frame of rat NTPDase2 have been described previously (Kegel B., et al., (1997) Neuropharmacology 36:1189-1200). Serum titers were determined by standard Western blot analysis under non-reducing conditions in the screening protein lysates from COS-7 cells expressing recombinant murine NTPDase1 or NTPDase2. The antibody specifically detected either NTPDase 1 or NTPDase2 of both mouse and rat tissues in immunohistochemical analysis.

Immunoblotting Procedures

Proteins were fractionated by sodium dodecyl sulfate (SDS)-polyacrylamide getl electrophoresis (Laemmli UK (1970) Nature 227:680-685). Protein samples were boiled in sample buffer (2% w/v SDS, 10% v/v glycerin, 0.001% bromophenol blue in 65 mM Tris, pH 6.8) under non-reducing conditions. The proteins were separated on a 10% acrylamide SDS-gel and transferred to Immobilon-P membrane (Millipore, Bedford, Mass.) by semi-dry electroblotting. Following incubation with the rabbit anti-NTPDase polyclonal antibodies, bands were visualized using horseradish peroxidase-conjugated goat anti-rabbit IgG at a dilution of 1:4000, and the Renaissance Chemiluminescence Reagent Plus, according to manufacturer's instructions.

Immunohistochemistry

Tissues were harvested, embedded in Triangle Biomedical Sciences (TBS) tissue freezing medium, snap-frozen in isopentane cooled on liquid nitrogen, and stored at −80° C. Six micrometer serial cryostat sections were fixed on ice cold acetone for 10 minutes and rinsed in phosphate-buffered saline (PBS) IgG binding sites were blocked with appropriate control serum diluted 1:5 In solution 9 (PBS, pH 7.4, supplemented with 0.1% bovine serum albumin, 150 mM tranexamic acid, 20 ug/mL aprotinin, 1.8 mM ethylenediaminetetraacetic acid, and 2 mM iodoacetic acid) further supplemented with 2% 3-omega fatty acid for one hour at room temperature. Sections were then incubated with primary antibody for one hours at room temperature (biotinylated NTPDase2-purified IgGs were incubated for 2 hours at 37° C.), rinsed in PBS, and incubated with 3% H₂O₂ in methanol for 5 minutes to deplete endogenous peroxidase. After incubation with the appropriate biotinylated IgG or F(ab′)₂ fragment of IgG for 30 minutes, staining was performed using the Vectastain ABC elite kit, with diaminobenzidine as the peroxidase substrate. Sections were counterstained lightly with Mayer's hematoxylin, dehydrated, cleared in xylene, and mounted with Permount. Detection of mouse antigens with mouse mAbs was performed as previously described (Sundberg C et al., (2001) Am. J Pathol. 158:1145-1160). All antibodies were diluted in solution 9 unless indicated otherwise. Optimal antibody concentrations were determined by serial dilution.

Human Colonic Tissue Samples

Cryopreserved human colonic tissue samples were obtained from the tissue bank of the Massachusetts General Hospital, Center for the Study of Inflammatory Bowel Disease. Tissue samples were studied from patients who had undergone colectomy for ulcerative colitis or Crohn's disease. Areas of uninvolved colon were also studied from patients undergoing colectomy for colon cancer to serve as a negative control. Cryopreserved tissue samples were stored at −80° C.

DSS Model

Adult mice (55-75 day old) sex-matched hemizygous CD39+/−, CD39−/−, and CD39+/+ littermates were given 3-5% (w/v) DSS in drinking water for seven days to induce a pathological state reminiscent of acute ulcerative colitis. Mice were fasted on day 7, and sacrificed the next day. Tissue was collected for Haematoxylin and Eosin staining, immunohistochemistry staining (for CD39, CD39L1, P-selectin, Fibrin, and P2Y1, for example), Western blot analysis, and NTPDase activity. To induce chronic ulcerative colitis, mice were treated with 3% DSS for seven days followed by a one week rest period. DSS treatment was resumed for seven days followed by another rest period of seven days. Treatment was resumed in the fifth week for seven days. At least six age- and sex-matched hemizygous CD39+/−, CD39−/−, and CD39+/+ mice were studied at the end of seven days of DSS treatment (acute colitis) or 5 week DSS treatment (chronic colitis). Mice were housed in standard cages and were allowed to drink and feed ad libitum. Animals were weighed daily.

Histologic Scoring of Crypt Damage and Inflammation

The distal colon was evaluated because this is the most severely affected colonic segment in DSS-induced colitis. Sections were routinely scored for crypt damage using Haematoxylin and Eosin stains.

Statistics

Where appropriate, data are expressed as the mean±SD. Groups were compared using the Student T test. Differences between experimental groups and controls were considered significant for P<0.05.

The following examples are meant to illustrate the invention. They are not meant to limit the invention in any way.

### EXAMPLE 1

Dysregulation of Extracellular Nucleotide Hydrolysis is Associated with Gastrointestinal Inflammation.

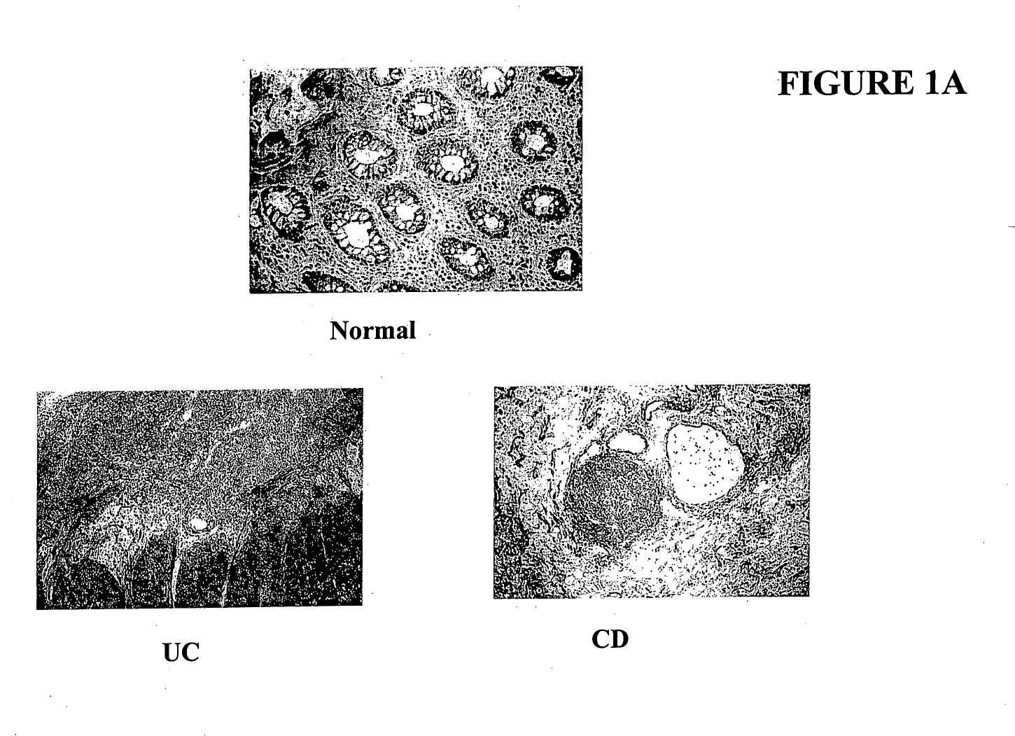

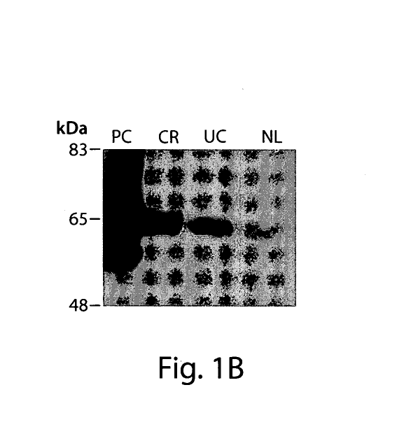

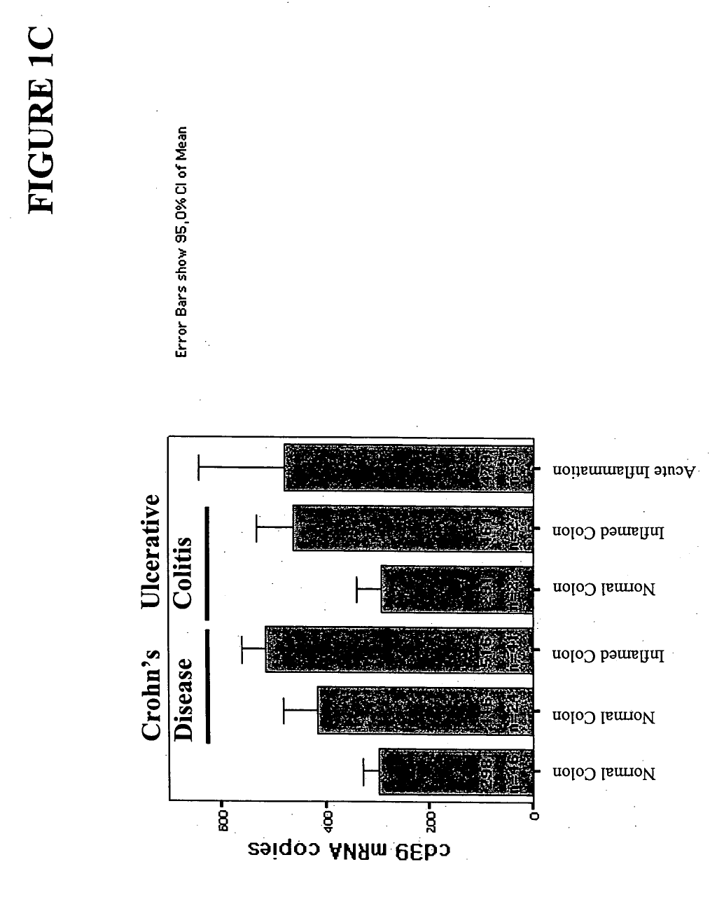

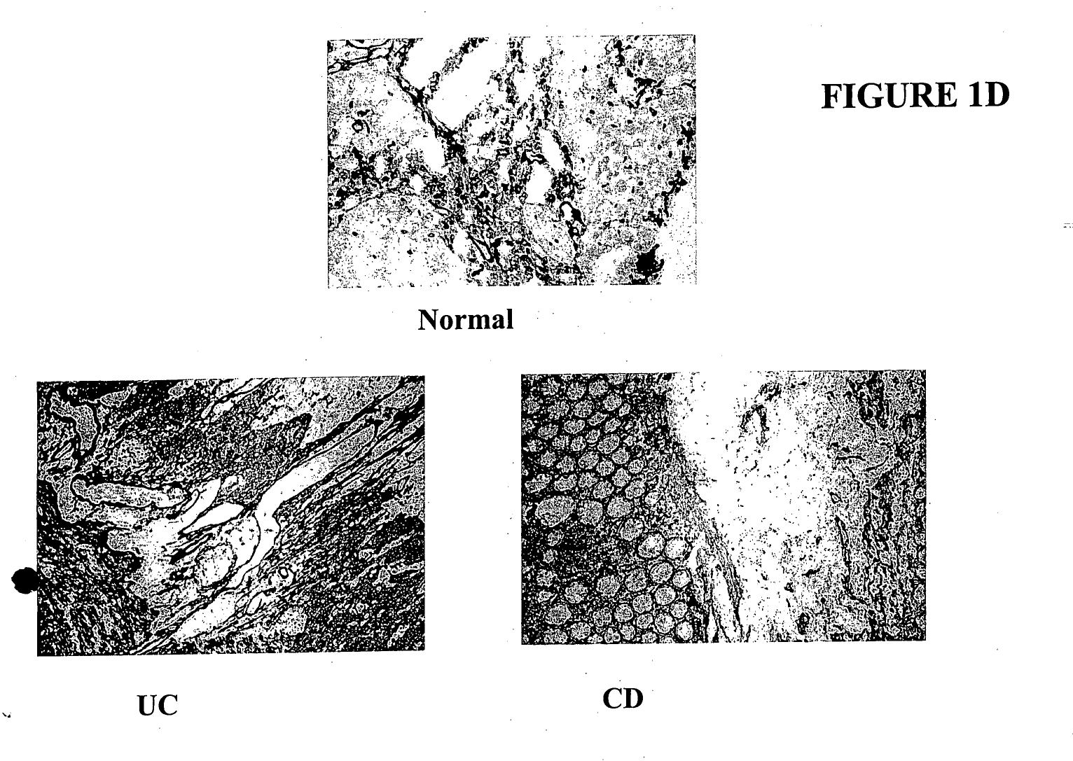

To investigate the role of NTPDase, such as CD39, in the development of the autoimmune condition known as intestinal bowel disease, colonic tissue samples were obtained endoscopically and surgically from areas of uninvolved colon from patients who underwent a colectomy for colon cancer, and patients with Crohn's disease (CD) and ulcerative colitis (UC). FIG. 1A shows the loss of integrity to the tissue architecture in tissue samples of patients diagnosed with CD and UC compared to normal patient. The loss in crypt structures in such tissues, as well as, the infiltration of immune cells, such as macrophages, monocytes, and eosinophils are characteristics of such conditions. UC samples were further characterized by the presence of new blood vessels (angiogenesis). Lymphoid aggregates were also present in CD samples, which is suggestive of a granuloma. CD39 localization and expression was next determined by immunohistochemistry (IH) (FIG. 1D) and Western Blot (FIG. 1B) analysis using a monoclonal antibody raised against CD39 cDNA (BU61). IH analysis of human colonic tissue revealed increased expression of CD39 staining intensity in both CD and UC patients relative to control patients. Whereas CD39 staining was observed mainly in perivascular regions in tissue samples from normal patients, UC and CD samples had significant stromal staining, indicative of tissue infiltration with smooth muscle cells, and inflammatory cells. Marked angiogenesis with new vessel staining for CD39 was also observed in the tissue specimens of patients with UC. Western Blot analysis further showed an up-regulation of CD39 expression in the colonic tissue of patients with UC (n=7) and CD (n=8) relative to control patients. The up-regulated expression of CD39 was intermediate in patients with ulcerative colitis and highest in patients with Crohn's disease. CD39 mRNA levels in ulcerative colitis and Crohn's disease were further measured by quantitative RT-PCR (FIG. 1C). While normal colonic tissue isolated from both UC (n=24) and CD (n=41) patients had an induction in CD39 mRNA levels relative to normal colonic tissues from normal patients (n=46), this increase in CD39 mRNA levels was further enhanced in areas of the colon that displayed inflammation (CD, n=41;UC, n=27).

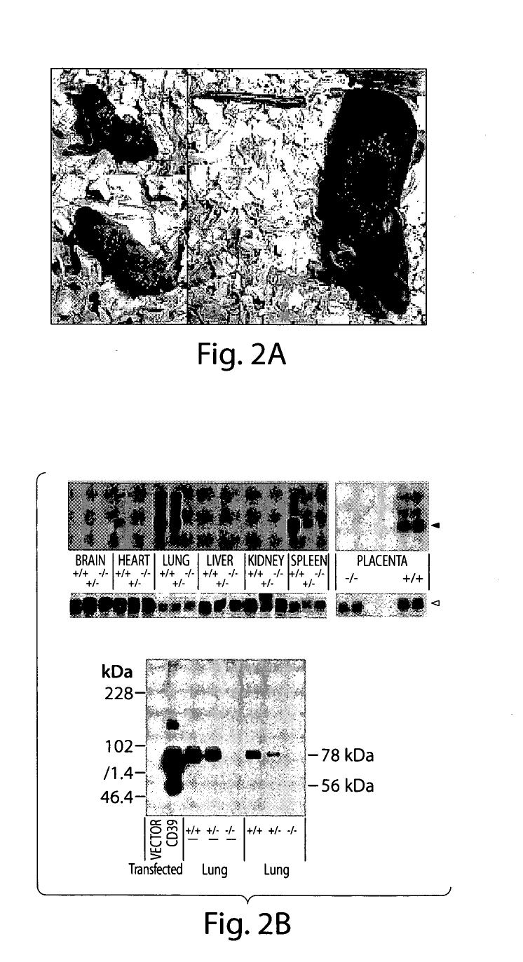

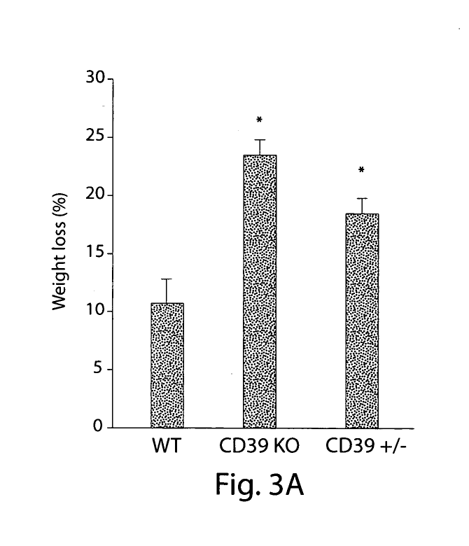

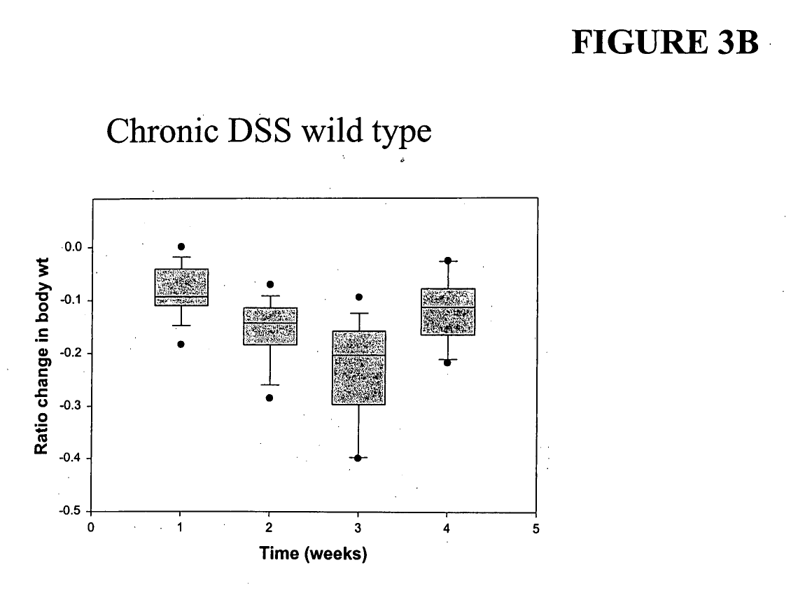

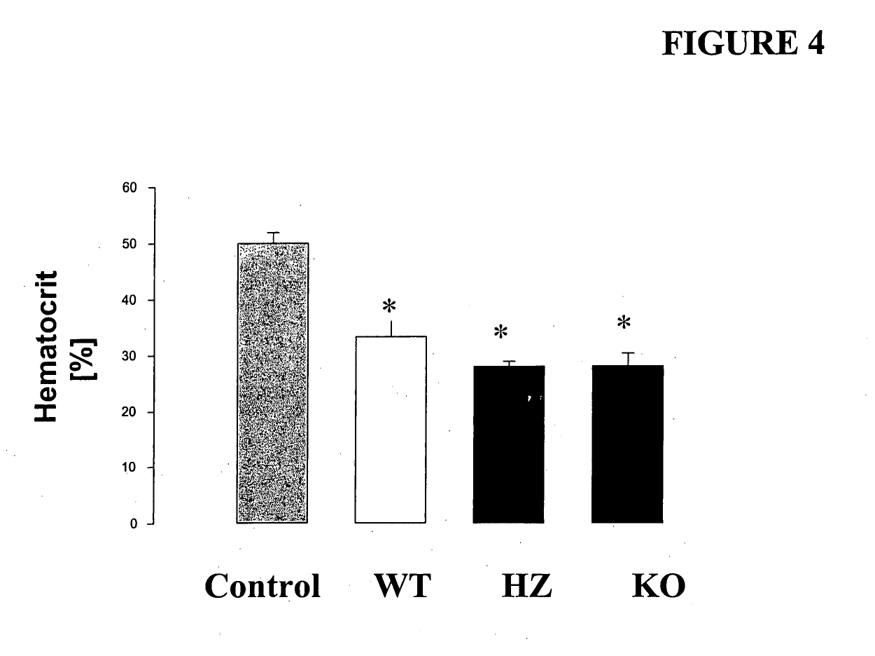

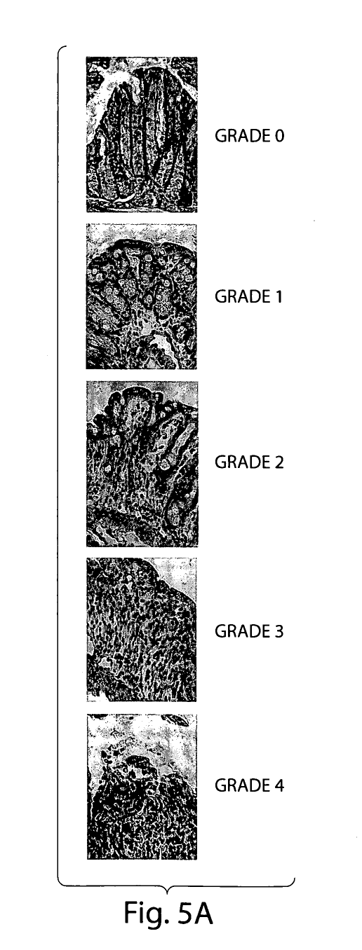

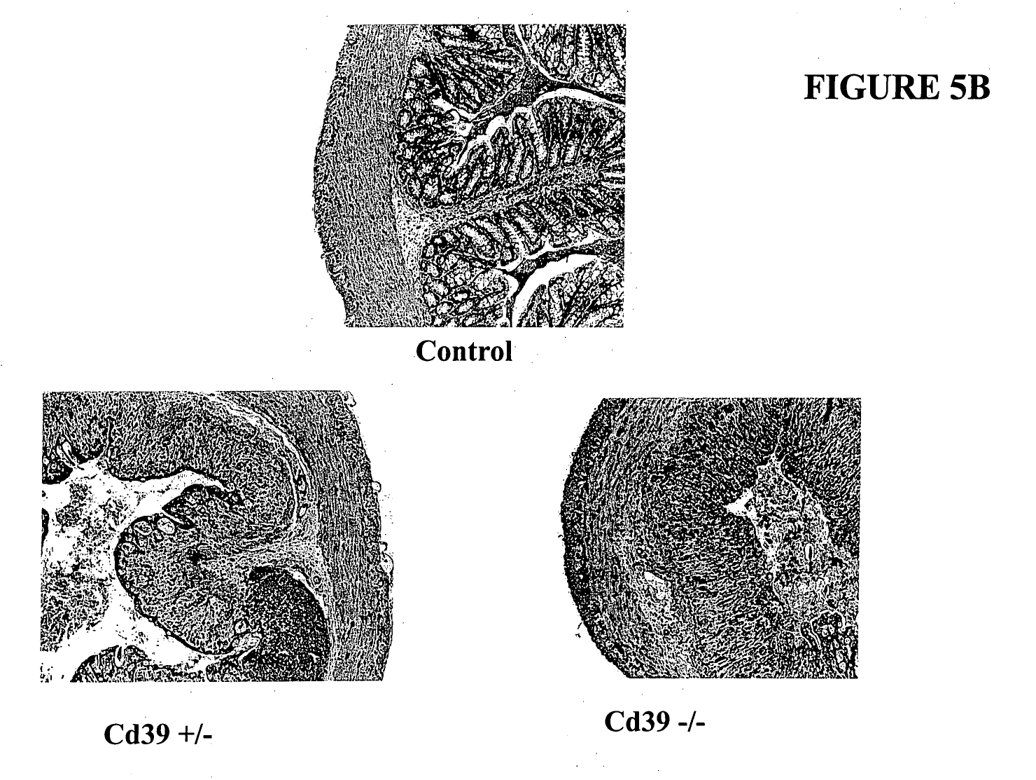

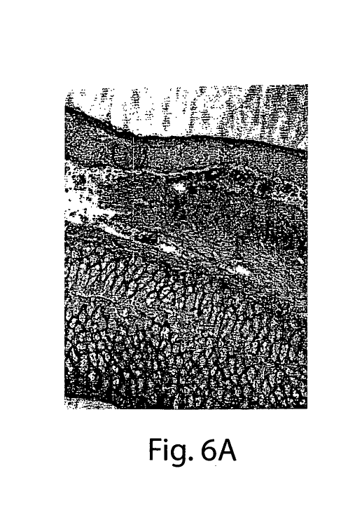

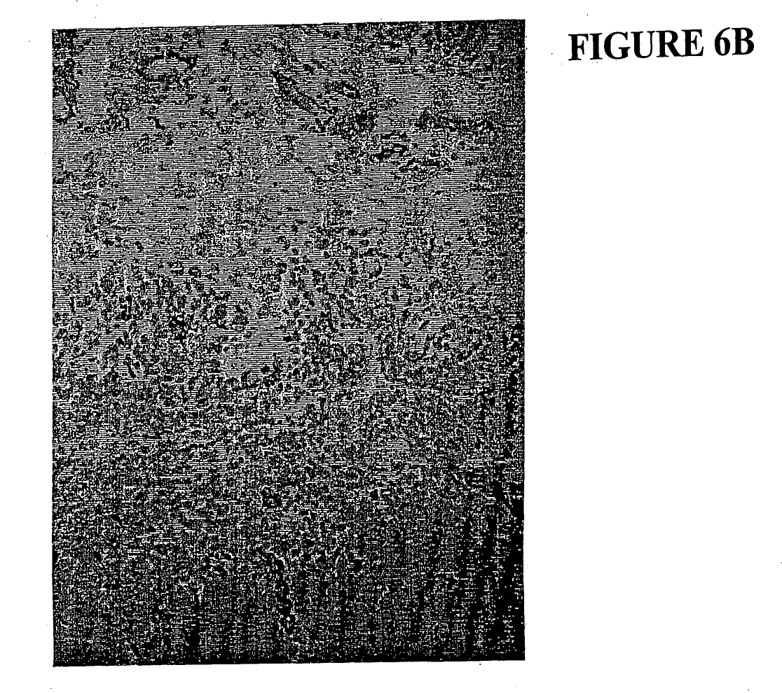

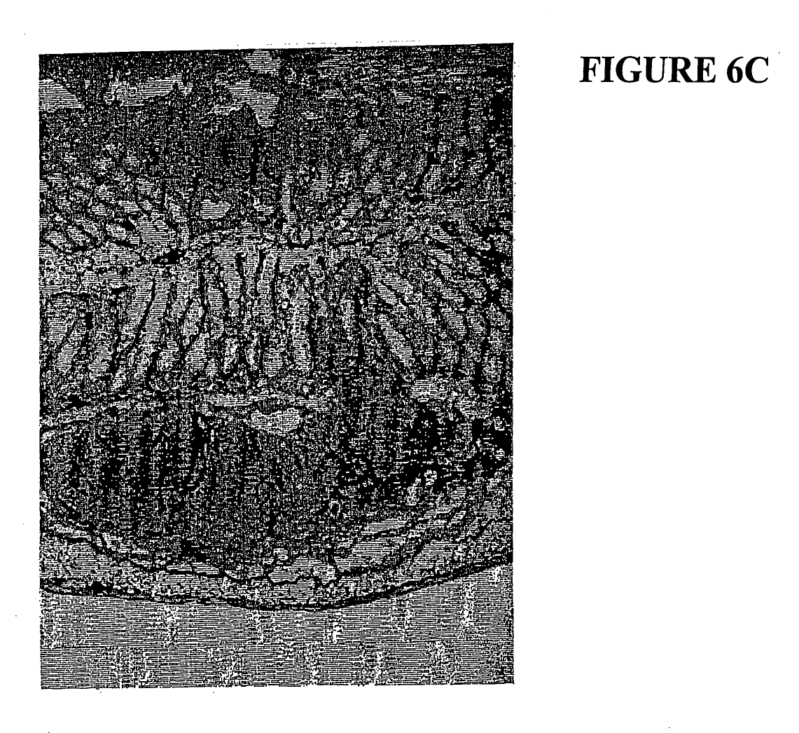

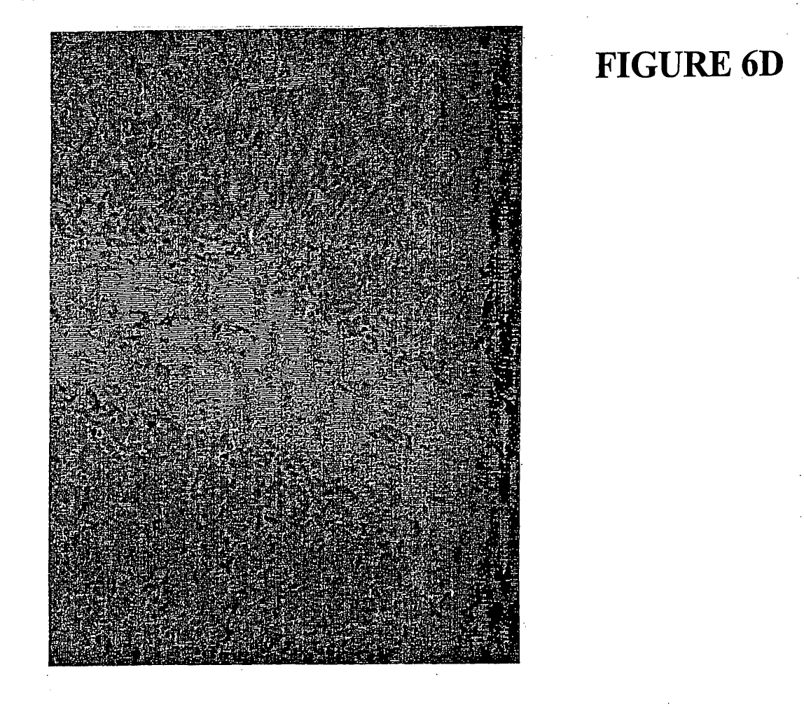

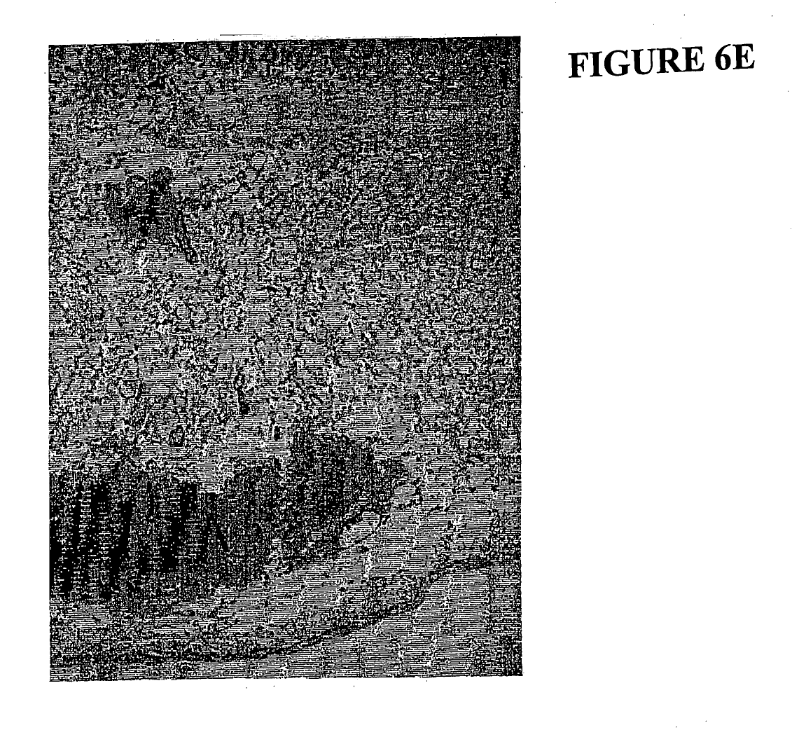

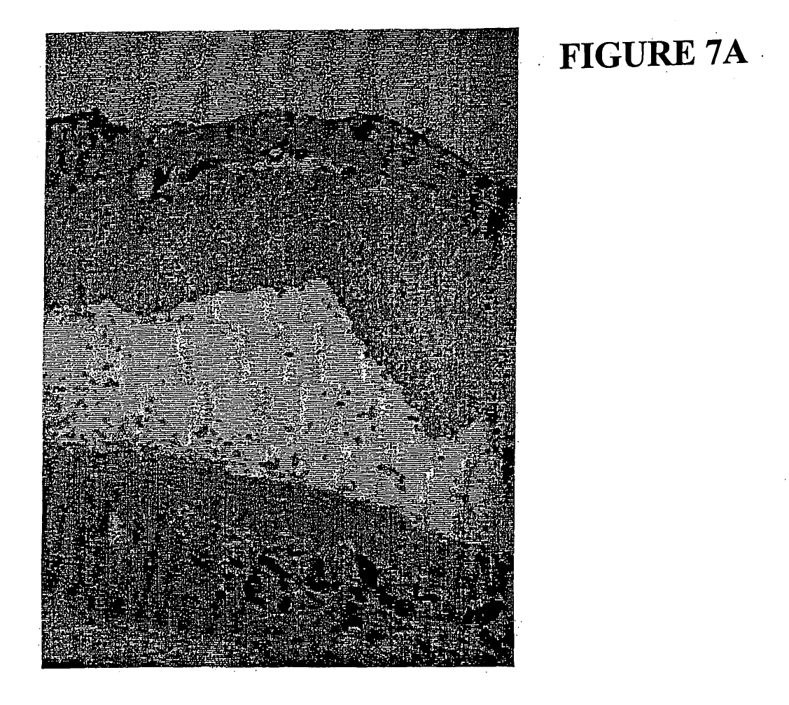

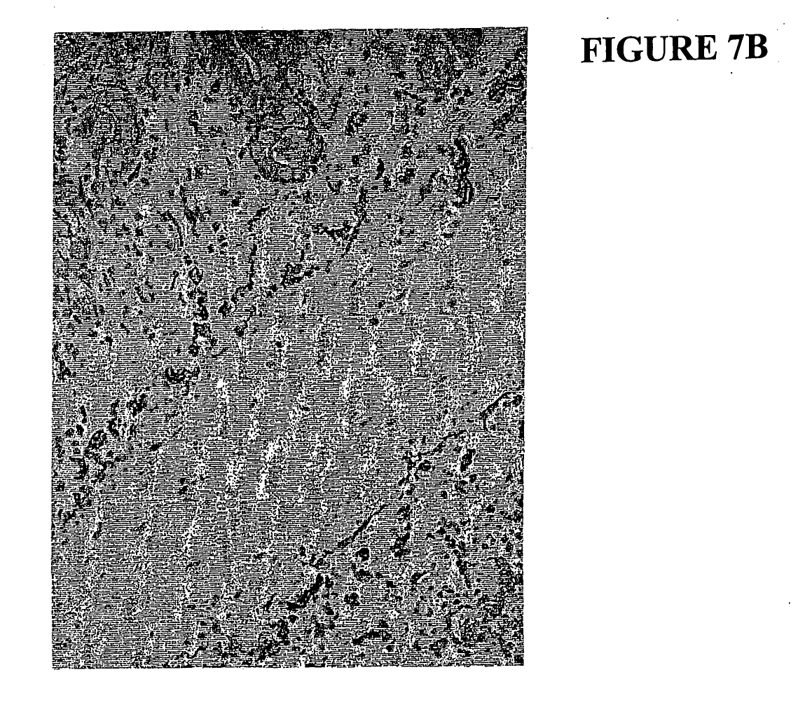

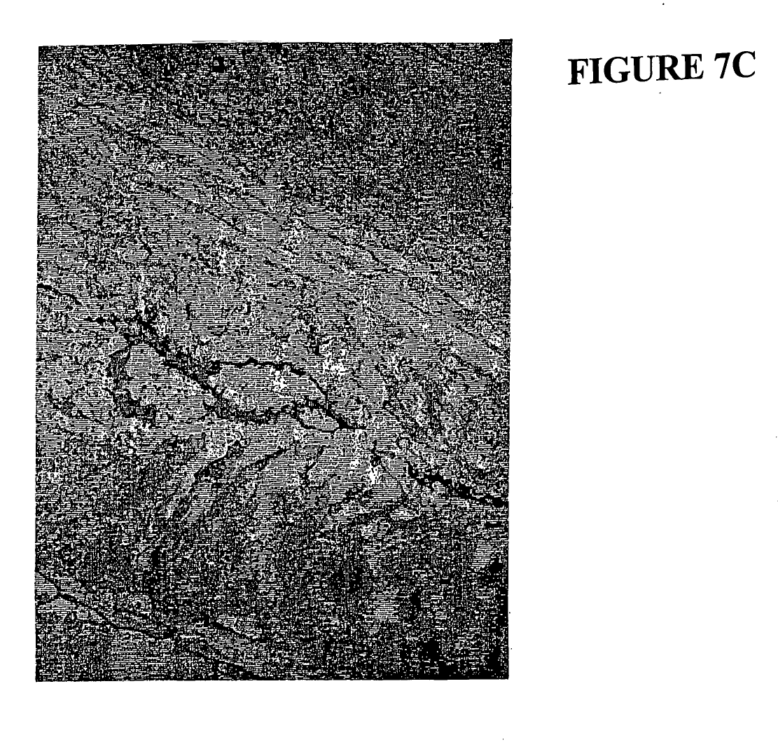

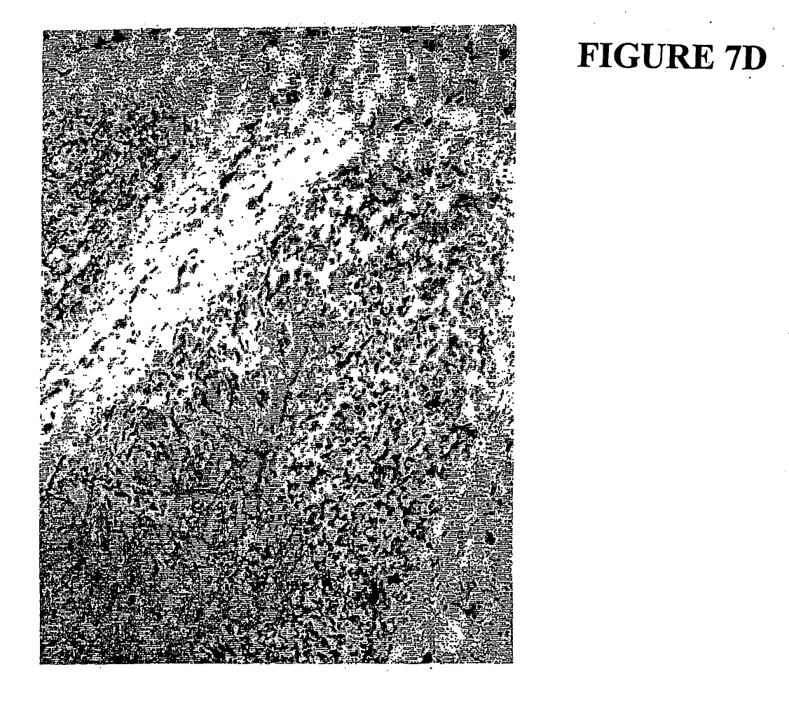

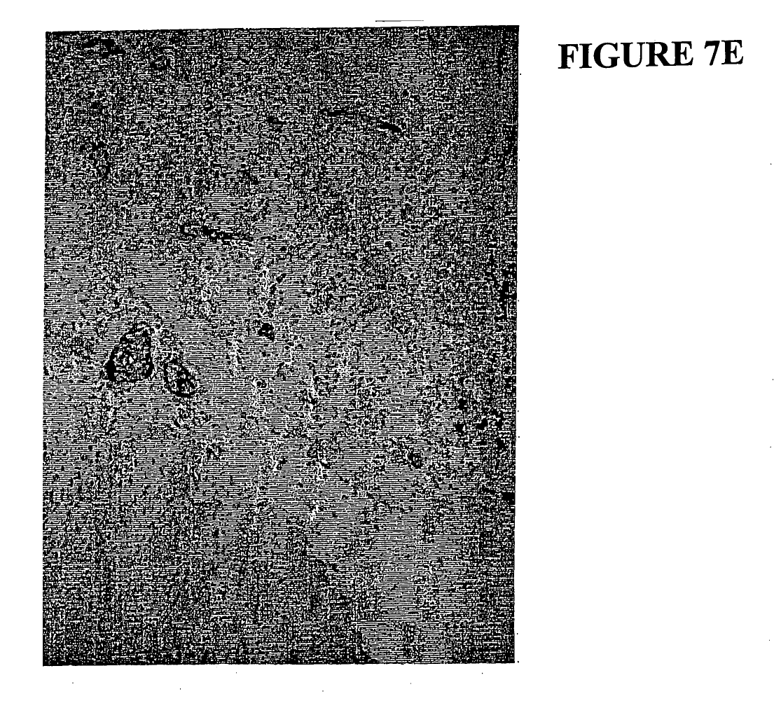

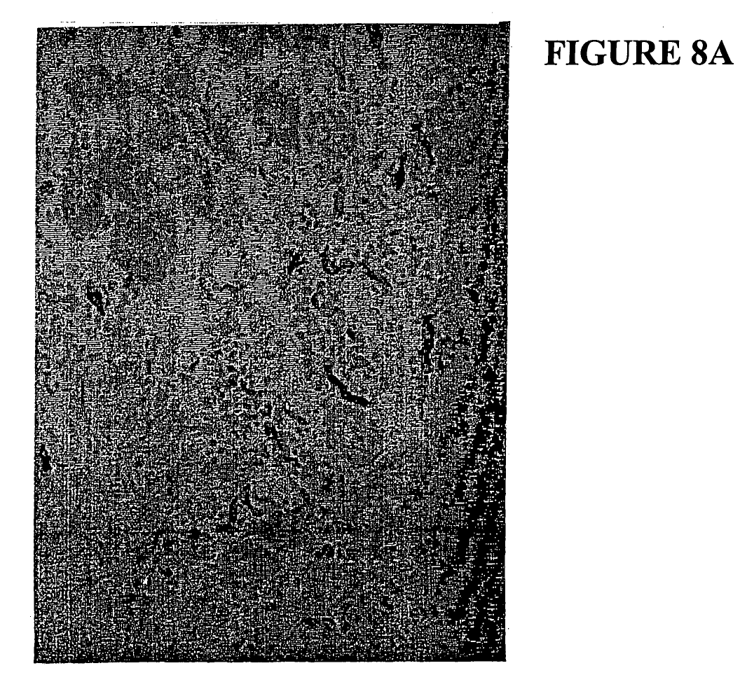

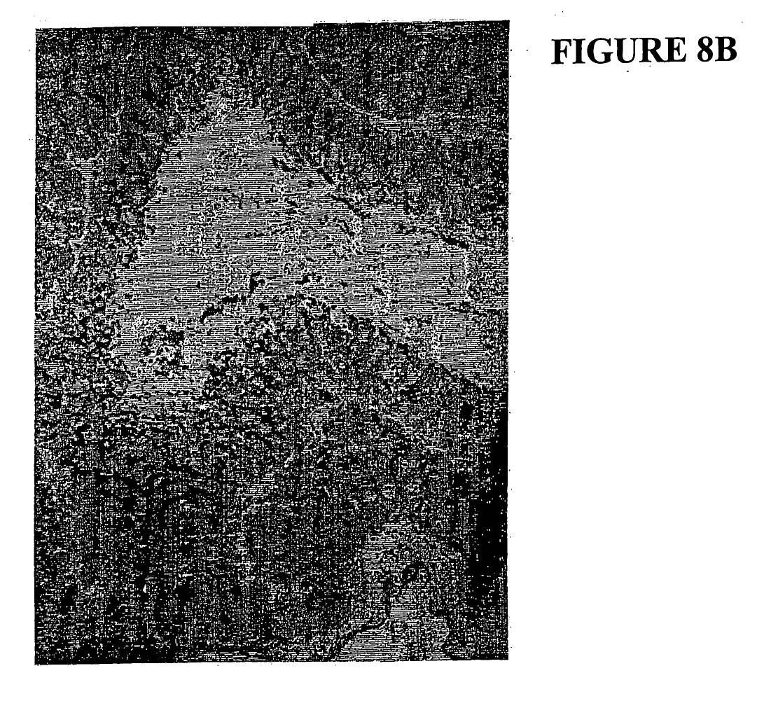

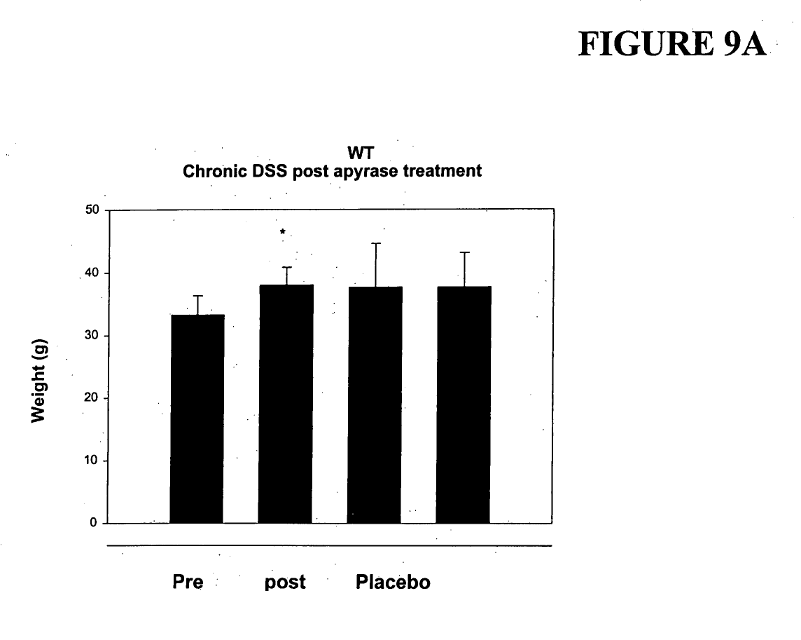

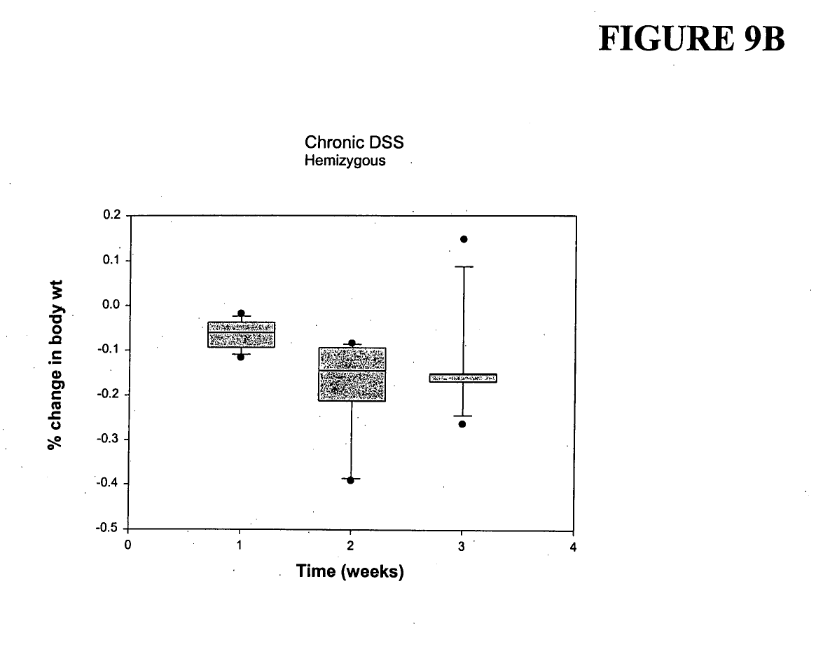

To confirm the above results and to investigate the cellular mechanisms involved in the mucosal injury observed in patients with UC and CD, we further used the dextran sulphate sodium (DSS)-induced experimental colitis in a mouse model. The generation of cd39-null mice has previously been described (see FIGS. 2A and 2B) (Enjyoji et al, Nature medicine, 1999; Imai et al, Molec. Med. 2000; Goepfert et al, Circulation 2001, Mizumoto et al, Nature Med 2002). CD39−/− mice, hemizygous CD39+/− mice, and wild-type mice CD39+/+ were given a 3-5% DSS aqueous solution orally for 7 days to induce colitis experimentally, and the consequent mucosal damage to the colonic tissue was evaluated macrocospically and histologically. The following criteria were assessed daily: weight, blood detection in stools by hemoccult, and stool consistency. FIG. 3A shows that DSS treatment resulted in 10% weight loss in wild type CD39+/+ mice when compared with approximately 25% in −/− and +/− mice, which is consistent with IBD in humans. Similar weight loss were further observed in chronic models of colitis in CD39+/+ mice over a period of five weeks (induced by the treatment of DSS for a week followed by a rest period of a week for an overall period of five weeks). FIG. 4 shows hematocrits measurements from CD39+/+ treated or not treated with DSS, as well as CD39+/− and CD39−/− mice which were treated with DSS. The reduction in red blood cell concentrations in mice treated with DSS, particularly in cases in which CD39 was deleted, is consistent with the development of hemorrhages and anemic episodes that typically occur in patients having IBD. Furthermore, as shown in FIG. 5B, CD39−/− mice developed a severe immune response relative to mice expressing CD39, characterized by mucosal ulceration, edema, hemorrhage, hyperemia, distorted crypt structures (using the crypt score of FIG. 5A), hyperplastic epithelium, ulcer healing by reepithelialization, and infiltration with mononuclear and polynuclear leukocytes in the lamina propria and submucosa. These changes were significantly attenuated in the hemizygous CD39−/+ and wild-type (+/+) mice. Furthermore, we observed in the CD39−/− mice that older mice are markedly more susceptible to DSS-induced experimental colitis than younger mice as evident by their 100% mortality within a week of exposure to DSS. We further confirmed this analysis by detecting levels of CD39, CD31 (endothelial cell marker), fibrin (to assess fibrin deposition), and P-selectin (platelet cell marker), as well as the presence of macrophages (marker of acute inflammation), in CD39+/+, CD39+/−, and CD39−/− mice following DSS treatment (see FIGS. 6A-6E, 7A-7E, 8A, and 8B). As expected, CD39 expression was strongest in tissue sections obtained from CD39+/+ mice, particularly in endothelial cells and in the stroma. Based on the expression of CD31 (an endothelial cell marker) and macrophage staining, angiogenesis and acute inflammation were both determined to be the most abundant in CD39−/− mice, relative to hemizygous (intermediate) and CD39−/− (lowest) mice. This experimental model of colitis shows that CD39 deficiency causes mice to become more susceptible to acute DSS-induced colitis, suggesting that CD39 might play an important regulatory role in the development of experimental as well as clinical IBD, including colitis. Our results also indicate that CD39 expression modulates innate immunity, thereby exerting a protective effect against experimental colitis.

### EXAMPLE 2

Administration of Apyrase, a NTPDase, Delays Weight Loss in Colitis

To test the hypothesis that the administration of an NTPDase can delay the damage caused by colitis, CD39+/+ and CD39+/− mice, following the induction of chronic colitis by DSS treatment, were administered with apyrase, an NTPDase (see FIGS. 4A and 4B). Apyrase was administered at 0.4 units/g weight at week five following the initial treatment with DSS. CD39+/+ mice showed a significant gain in weight following apyrase treatment, and this effect was further enhanced in CD39−/+ mice. These results therefore indicate that the damage caused by immune responses in autoimmune conditions such as colitis can be reduced by the administration of an NTPDase.

### Other Embodiments

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

1. A method of diagnosing a mammal having or at risk of having an autoimmune condition, wherein said a reduction in NTPDase biological activity identifies said mammal as having or at risk of having said condition.

2. The method of claim 1, wherein said autoimmune condition is Addison's disease, alopecia, ankylosing spondylitis, antiphospholipid syndrome, Behcet's disease, chronic fatigue syndrome, Crohn's disease, ulcerative colitis, diabetes, fibromyalgia, Goodpasture syndrome, Graves' disease, idiopathic thrombocytopenic purpura, lupus, Meniere's multiple sclerosis, myasthenia gravis, pemphigus vulgaris, primary biliary cirrhosis, psoriasis, rheumatoid arthritis, rheumatic fever, sarcoidosis, scleroderma, vasculitis, vitiligo, or Wegener's granulomatosis.

3. The method of claim 1, wherein said reduction in said level of NTPDase activity is a reduction in the level of NTPDase mRNA, NTPDase protein, or the phosphohydrolytic activity of NTPDase.

4. A method of diagnosing a mammal having or at risk of having an autoimmune condition, wherein said an increase in P2 receptor biological activity identifies said mammal as having or at risk of having said condition.

5. The method of claim 4, wherein said autoimmune condition is Addison's disease, alopecia, ankylosing spondylitis, antiphospholipid syndrome, Behcet's disease, chronic fatigue syndrome, Crohn's disease, ulcerative colitis, diabetes, fibromyalgia, Goodpasture syndrome, Graves' disease, idiopathic thrombocytopenic purpura, lupus, Meniere's multiple sclerosis, myasthenia gravis, pemphigus vulgaris, primary biliary cirrhosis, psoriasis, rheumatoid arthritis, rheumatic fever, sarcoidosis, scleroderma, vasculitis, vitiligo, or Wegener's granulimatosis.

6. The method of claim 4, wherein said increase in said level of P2 receptor activity is an increase in the level of P2 receptor MRNA, P2 receptor protein, or the biological activity of the P2 receptor.

7. A method for identifying a candidate compound for treating, reducing, or preventing an autoimmune condition in a mammal, said method comprising:
(a) contacting a cell expressing a NTPDase gene with a candidate compound; and
(b) measuring NTPDase gene expression or NTPDase protein activity in said cell, a candidate compound that increases said expression or said activity, relative to NTPDase expression or activity in a cell not contacted with said candidate compound, identifying said candidate compound as a candidate compound useful for treating, reducing, or preventing an autoimmune disorder in a mammal.

8. The method of claim 7, wherein said NTPDase gene is a NTPDase fusion gene.

9. The method of claim 7, wherein step (b) comprises measuring expression of NTPDase MRNA or protein.

10. The method of claims 7, wherein said NTPDase is CD39.

11. The method of claim 7, wherein said cell is a mammalian cell.

12. The method of claim 11, wherein said cell is a rodent cell.

13. A method for identifying a candidate compound for treating, reducing, or preventing an autoimmune condition in a mammal, said method comprising:
(a) contacting a cell expressing a P2 receptor gene with a candidate compound; and
(b) measuring P2 receptor gene expression or P2 receptor activity in said cell, a candidate compound that decreases said expression or said activity, relative to P2 receptor expression or activity in a cell not contacted with said candidate compound, identifying said candidate compound as a candidate compound useful for treating, reducing, or preventing said autoimmune disorder in a mammal.

14. The method of claim 13, wherein said P2 receptor gene is a P2 receptor fusion gene.

15. The method of claim 13, wherein step (b) comprises measuring expression of P2 receptor mRNA or protein.

16. The method of claim 13, wherein said cell is a mammalian cell.

17. The method of claim 16, wherein said cell is a rodent cell.

18. A method for identifying a candidate compound for treating, reducing, or preventing an autoimmune disorder in a mammal, said method comprising:
(a) contacting NTPDase protein with a candidate compound; and
(b) determining whether said candidate compound binds said NTPDase protein, a candidate compound that binds said NTPDase protein and increases the activity of NTPDase being a candidate compound useful for treating, reducing, or preventing said autoimmune disorder.

19. The method of claim 18, wherein said NTPDase is human NTPDase.

20. The method of claim 18, wherein said NTPDase is CD39.

21. A method for identifying a candidate compound for treating, reducing, or preventing an autoimmune disorder in a mammal, said method comprising:
(a) contacting P2 receptor with a candidate compound; and
(b) determining whether said candidate compound binds said P2 receptor, a candidate compound that binds said P2 receptor and decreases the activity of P2 receptor being a candidate compound useful for treating, reducing, or preventing an autoimmune disorder.

22. The method of claim 21, wherein said P2 receptor is human P2 receptor.
